(19) United States
(12) Patent Application Publication
(10) Pub. No.: US 2012/0125332 A1
(43) Pub. Date: May 24, 2012

(54) APPARATUS, SYSTEMS, AND METHODS FOR RESPIRATORY THERAPY

(75) Inventors: William F. Niland, Arnold, MD (US); Felino V. Cortez, JR., Bowie, MD (US)

(73) Assignee: Vapotherm, Inc., Stevensville, MD (US)

(21) Appl. No.: 13/246,226

(22) Filed: Sep. 27, 2011

Related U.S. Application Data
(60) Provisional application No. 61/415,477, filed on Nov. 19, 2010.

(51) Int. Cl. A61M 16/16; A61M 16/00

(52) U.S. Cl. 128/203.16; 128/207.18

(57) ABSTRACT

A nasal cannula for administering respiratory therapy is disclosed. The nasal cannula comprises first and second supply tubes, a cannula body, first and second nasal prongs, and first and second flow paths. Each supply tube has an inlet to receive a flow from a flow source and an outlet. The cannula body has first and second ends. The first end is coupled to the first outlet and the second end is coupled to the second outlet. The first and second nasal prongs extend from the cannula body. The first flow path extends from the first supply tube through the cannula body to the first nasal prong. The second flow path extends from the second supply tube through the cannula body to the second nasal prong. Systems and methods for using the above-described nasal cannula are also disclosed.

### CROSS-REFERENCE TO RELATED APPLICATIONS

This application claims priority to U.S. Patent Application No. 61/415,477, entitled “APPARATUS, SYSTEMS, AND METHODS FOR RESPIRATORY THERAPY,” filed on Nov. 19, 2010, the contents of which are incorporated herein by reference in their entirety.

### FIELD OF THE INVENTION

The present invention relates generally to respiratory therapy, and more particularly to nasal cannulas.

### BACKGROUND OF THE INVENTION

Conventional respiratory therapy involves administering either a flow of breathing gas or a medicament to a patient's respiratory system.

To administer a flow of breathing gas, nasal cannulas are often used. A nasal cannula provides breathing gases directly to a user's respiratory system via nasal inhalation. A nasal cannula typically includes two prongs, with each prong inserted into a respective nostril during use. The nasal cannula may be retained during use by looping tubing attached to the cannula over the user's ears and drawing the tubing tight under the user's chin. A conventional nasal cannula is described in U.S. Patent Application Publication No. US 2008/0121230 A1.

To administer a drug to the user's respiratory system, nebulizers are generally used. A nebulizer converts a conventional liquid medicament into a fine mist for inhalation by a patient. The mist may include a medicament for delivery to the respiratory tract of the patient.

Improved apparatus, systems, and methods for respiratory therapy are desired.

### SUMMARY OF THE INVENTION

Aspects of the present invention are directed to apparatus, systems, and methods for administering respiratory therapy.

In accordance with one aspect of the present invention, a nasal cannula for administering respiratory therapy to a user is disclosed. The cannula comprises a first supply tube, a second supply tube, a cannula body, first and second nasal prongs, a first flow path, and a second flow path. The first supply tube has a first inlet to receive a first flow from a first flow source and a first outlet. The second supply tube has a second inlet to receive a second flow from a second flow source and a second outlet distinct from the first inlet and the first outlet, respectively. The cannula body has first and second ends. The first end is coupled to the first outlet and the second end is coupled to the second outlet. The first and second nasal prongs extend from the cannula body. The first flow path extends from the first supply tube through the cannula body to the first nasal prong to deliver at least a portion of the first flow through the first nasal prong. The second flow path extends from the second supply tube through the cannula body to the second nasal prong to deliver at least a portion of the second flow through the second nasal prong.

In accordance with another aspect of the present invention, a system for administering respiratory therapy to a user is disclosed. The system comprises the above-described nasal cannula, a first flow source, and a second flow source.

In accordance with yet another aspect of the present invention, a method for administering respiratory therapy to a user is disclosed. The method comprises the steps of positioning the above-described nasal cannula on a user such that the first and second nasal prongs are positioned in respective nostrils of the user, providing a flow of heated and humidified gas along the first flow path; and providing a flow of aerosolized medicament separate from the flow of heated and humidified gas along the second flow path.

### BRIEF DESCRIPTION OF THE DRAWINGS

The invention is best understood from the following detailed description when read in connection with the accompanying drawings, with like elements having the same reference numerals. When a plurality of similar elements are present, a single reference numeral may be assigned to the plurality of similar elements with a small letter designation referring to specific elements. When referring to the elements collectively or to a non-specific one or more of the elements, the small letter designation may be dropped. This emphasizes that according to common practice, the various features of the drawings are not drawn to scale unless otherwise indicated. On the contrary, the dimensions of the various features may be expanded or reduced for clarity. Included in the drawings are the following figures:

FIG. 1 is a diagram illustrating an exemplary nasal cannula for providing respiratory therapy to a user in accordance with aspects of the present invention;

FIG. 2 is an image of the nasal cannula of FIG. 1;

FIG. 3 is a diagram illustrating an exemplary system for providing respiratory therapy to a user in accordance with aspects of the present invention;

FIG. 4 is an image of the system of FIG. 3; and

FIG. 5 is a flowchart illustrating an exemplary method for providing respiratory therapy to a user in accordance with aspects of the present invention.

### DETAILED DESCRIPTION OF THE INVENTION

The exemplary apparatus, systems, and methods disclosed herein relate to a dual-inlet nasal cannula. The exemplary embodiments disclosed herein are usable, for example, to deliver high-flow heated and humidified gas and an aerosolized medicament simultaneously to promote respiratory therapy.

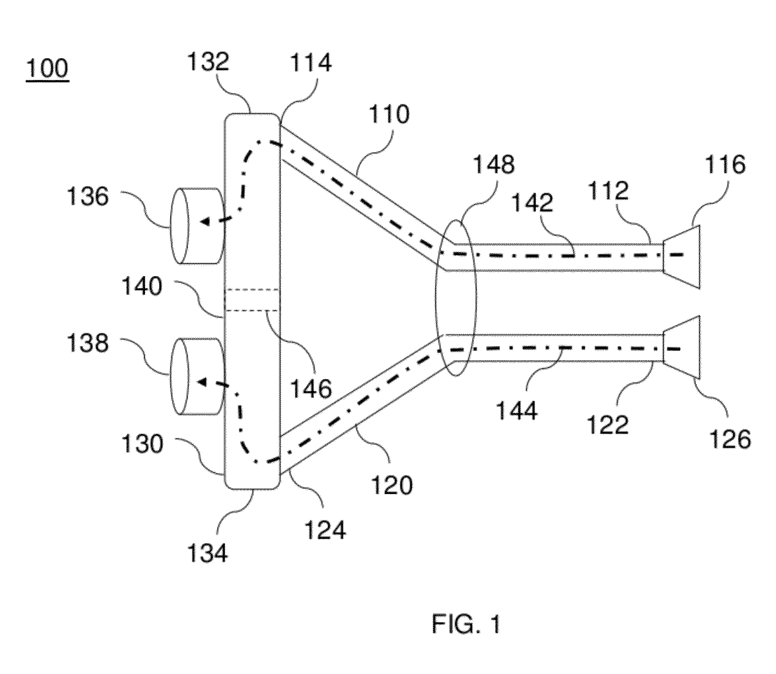

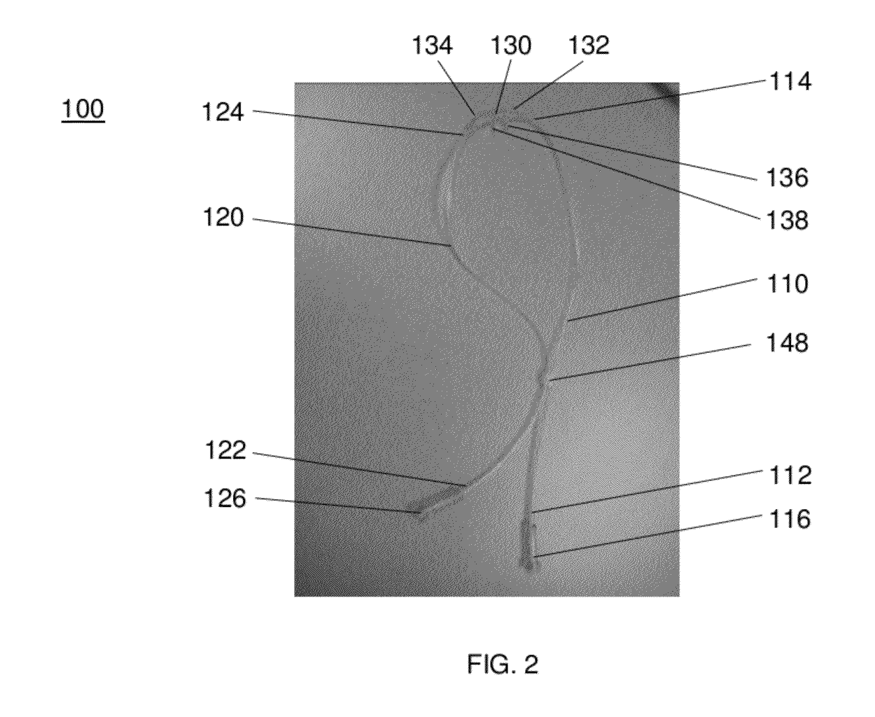

Referring now to the drawings, FIGS. 1 and 2 illustrate an exemplary nasal cannula 100 for administering respiratory therapy to a user in accordance with aspects of the present invention. Nasal cannula 100 is usable in conjunction with separate flow sources, e.g. of breathing gas or medicine, to promote respiratory therapy. Generally speaking, nasal cannula 100 includes a first supply tube 110, a second supply tube 120, and a cannula body 130. Additional details of nasal cannula 100 are provided herein.

Supply tube 110 is an elongated hollow lumen. As shown in FIG. 1, supply tube 110 defines an inlet 112 at one end of the lumen and an outlet 114 at an opposite end of the lumen. Supply tube 110 enables fluid communication between the inlet 112 and outlet 114 through the lumen. Suitable supply tubes for use as supply tube 110 will be known to one of ordinary skill in the art from the description herein.

Supply tube 120 is also an elongated hollow lumen, substantially as described above with respect to supply tube 110. As shown in FIG. 1, supply tube 120 defines an inlet 122 at one end of the lumen and an outlet 124 at an opposite end of the lumen. Supply tube 120 enables fluid communication between the inlet 122 and outlet 124 through the lumen. Inlet 122 and outlet 124 of supply tube 120 are distinct from inlet 112 and outlet 114 of supply tube 110.

Supply tubes 110 and 120 may further include connectors 116 and 126, respectively. As shown in FIGS. 1 and 2, connector 116 is coupled to the inlet 112 of supply tube 110, and connector 126 is coupled to the inlet 122 of supply tube 120. Connectors 116 and 126 are distinct from each other, i.e., each connector 116 and 126 is capable of mating with a separate component. This enables supply tubes 110 and 120 to receive flow from different flow sources via connectors 116 and 126, respectively, as will be explained herein. Connectors 116 and 126 may be separate components, as shown in FIGS. 1 and 2, or may be integrally formed into one component, so long as each connector 116 and 126 is capable of receive a flow from a distinct flow source. In an exemplary embodiment, connectors 116 and 126 are standard oxygen tubing connectors. Suitable connectors will be known to one of ordinary skill in the art from the description herein.

Cannula body 130 is coupled to supply tubes 110 and 120. Cannula body 130 has ends 132 and 134. End 132 of cannula body 130 is coupled to receive a flow from outlet 114 of first supply tube 110. End 134 of cannula body 130 is coupled to receive a flow from outlet 124 of second supply tube 120.

Cannula body 130 includes nasal prongs 136 and 138 extending from cannula body 130, as shown in FIG. 1. Nasal prongs 136 and 138 provide the flow received from supply tubes 110 and 120 to a user of nasal cannula 100.

In an exemplary embodiment, cannula body 130 comprises a tubular portion 140, as shown in FIGS. 1 and 2. Ends 132 and 134 comprise opposite axial ends of the tubular portion 140. Nasal prongs 136 and 138 extend outward from tubular portion 140 in a substantially radial direction. As shown in FIG. 2, supply tube 110 is coupled to provide a first flow to cannula body 130 via axial end 132, and supply tube 120 is coupled to provide a second flow to cannula body 130 via axial end 134.

The above components of nasal cannula 100 define a first flow path 142 and a second flow path 144 distinct from the first flow path. First flow path 142 extends from first supply tube 110 through cannula body 130 to first nasal prong 136. Second flow path 144 extends from second supply tube 120 through cannula body 130 to second nasal prong 138.

It will be understood by one of ordinary skill in the art that flow paths 142 and 144 are not limited to the above-defined destinations. Cannula body 130 may optionally include a septum 146. In an exemplary embodiment, septum 146 comprises a wall formed in the internal portion of cannula body 130, as shown in dashed lines in FIG. 1. Septum 146 is positioned between nasal prongs 136 and 138. Septum 146 seals one side of cannula body 130 from the other side of cannula body 130.

Where cannula body 130 does not include a septum, each of the nasal prongs 136 and 138 will be in fluid communication with both of the supply tubes 110 and 120. Thus, first flow path 142 may extend to both nasal prongs 136 and 138, and second flow path 144 may also extend to both nasal prongs 136 and 138. This may be desirable in order to enable each nostril to receive a mixed flow from both sources. This may further enable flows to be delivered to each nostril at the same concentration, and/or at the same temperature.

Conversely, where cannula body 130 includes septum 146, septum 146 blocks fluid communication between supply tube 110 and nasal prong 138, and between supply tube 120 and nasal prong 136. Thus, septum 146 blocks first flow path 142 from extending to nasal prong 138, and blocks second flow path 144 from extending to nasal prong 136. Septum 146 may be desirable so that a flow to one nasal prong 136 that has a relatively high back-pressure will not stop or slow a flow to the other nasal prong 138 that has a relatively low back-pressure. This may further prevent a change in temperature of one of the flows through contact with the other, thereby minimizing condensation build up in the body of the nasal cannula and avoiding the delivery of liquid droplets into the user's nose.

It will be understood that nasal cannula 100 is not limited to the above components, but may include additional or alternative components, as would be understood by one of ordinary skill in the art. For example, nasal cannula 100 may include a draw cuff 148 for keeping supply tubes 110 and 120 together and in place when nasal cannula 100 is worn by the user.

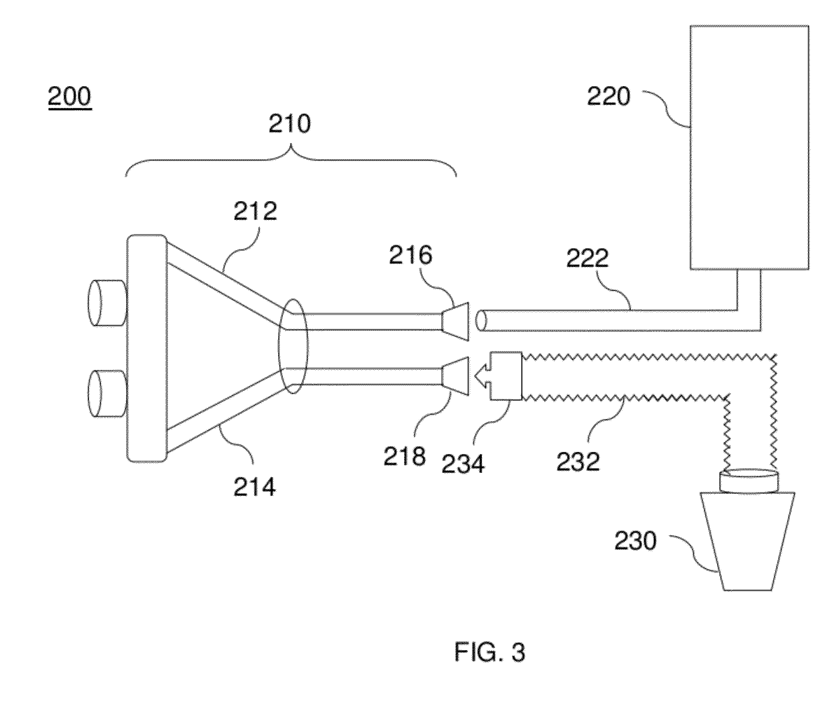

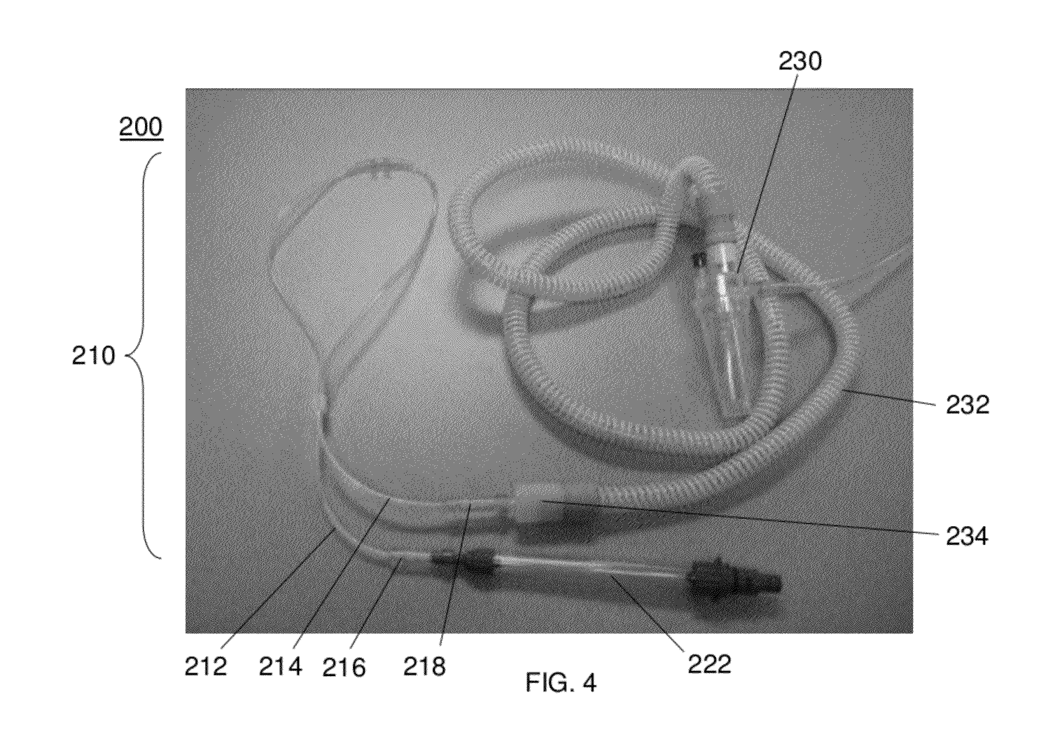

FIGS. 3 and 4 illustrate an exemplary system 200 for administering respiratory therapy to a user in accordance with aspects of the present invention. Generally speaking, system 200 includes a nasal cannula 210, a first flow source 220, e.g. of heated and humidified gas, and a second flow source 230, e.g. of aerosolized medicament. Additional details of system 200 are provided herein.

Nasal cannula 210 is a nasal cannula substantially as described with respect to exemplary nasal cannula 100. Nasal cannula 210 includes supply tubes 212 and 214. Supply tubes 212 and 214 may further include connectors 116 and 126, respectively. As shown in FIGS. 3 and 4, connector 216 is coupled to the inlet of supply tube 212, and connector 218 is coupled to the inlet of supply tube 214. Connectors 216 and 218 are distinct from each other, i.e. each connector 216 and 218 is capable of mating with a separate component. This enables supply tubes 212 and 214 to receive flows from different sources via connectors 216 and 218, respectively, and transmit the flows along different flow paths through cannula 210 to the user.

Source 220 generates heated and humidified gas. Source 220 is coupled to provide heated and humidified gas to the inlet of supply tube 212. In an exemplary embodiment, source 220 may include a delivery tube 222, as shown in FIGS. 3 and 4. Delivery tube 222 may be coupled to connector 216 in order to provide the heated and humidified gas from source 220 to the inlet of supply tube 212 and along a first flow path of cannula 210. Suitable sources 220 of heated and humidified gas will be known to one of ordinary skill in the art. For example, source 220 may be the Vapotherm 2000i, the Vapotherm Precision Flow, the Vapotherm Flowrest System, or the Vapotherm Careflow system provided by Vapotherm, Inc of Stevensville, Md., USA. Other suitable sources 220 will be known to one of ordinary skill in the art from the description herein.

Source 230 generates aerosolized medicament. Source 230 is coupled to provide the aerosolized medicament to the inlet of supply tube 214. In an exemplary embodiment, source 230 may include a corrugated tube 232, as shown in FIGS. 3 and 4. Corrugated tube 232 is coupled to connector 218 in order to provide the aerosolized medicament from source 230 to the inlet of supply tube 214 and along a second flow path of cannula 210. As shown in FIG. 3, corrugated tube 232 has a greater diameter than delivery tube 222 of source 220. This may be desirable in order to ensure that the aerosolized medicament remains in mist form in the gas from source 230. Additionally, source 230 may include a circuit connector 234 for coupling corrugated tube 232 to the connector 218 of supply tube 214. Suitable sources 230 of aerosolized medicament will be known to one of ordinary skill in the art. For example, source 230 may be any conventional nebulizer, such as AirLife Brand Misty Finity Continuous Nebulizers provided by Cardinal Health, Inc of Dublin, Ohio, USA. Other suitable sources 230 will be known from the description herein.

Nasal cannula 210 may optionally include a septum, as described above with respect to nasal cannula 100. Where nasal cannula 210 does not include a septum, then each flow path in cannula 210 may extend to both nasal prongs of cannula 210. Thus, the nasal prongs of nasal cannula 210 may receive both the heated and humidified gas from source 220 and the aerosolized medicament from source 230. Where nasal cannula 210 includes a septum, then each flow path in cannula 210 extends to only the proximate nasal prong of cannula 210. Thus, one nasal prong of nasal cannula 210 may receive the heated and humidified gas from source 220, and the other nasal prong of nasal cannula 210 may receive the aerosolized medicament from source 230.

While source 220 is described as providing heated and humidified gas, and source 230 is described as providing aerosolized medicament, it will be understood to one of ordinary skill in the art that these sources are not so limited. Other flows that may be provided by sources 220 and 230 include, for example, sources of gas such as heliox, inhaled nitric oxide (INO), 100% oxygen, room air, and/or air-oxygen mix.

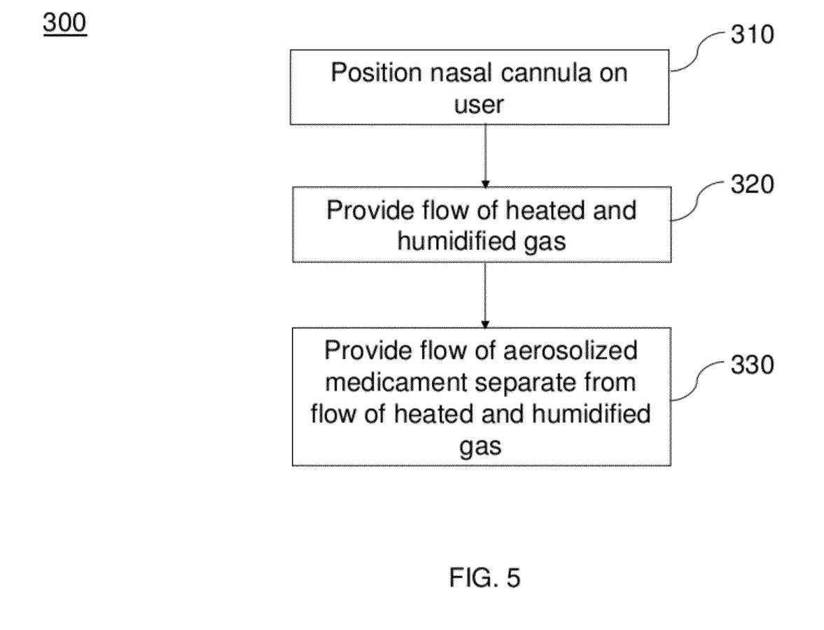

FIG. 5 is a flowchart illustrating an exemplary method 300 for providing respiratory therapy to a user in accordance with aspects of the present invention. Generally speaking, method 300 includes positioning a nasal cannula on a user, providing a flow of heated and humidified gas, and providing a flow of aerosolized medicament. Additional details of method 300 are provided herein. For the purposes of illustration, the steps of method 300 are described herein with reference to the exemplary system 200 described above.

In step 310, a nasal cannula is positioned on a user. In an exemplary embodiment, nasal cannula 210 is positioned on a user such that first and second nasal prongs are positioned in the nostrils of the user. For example, the user may position the nasal cannula 210 on his or herself. Alternatively, a care provider may position the nasal cannula 210 on the user. Nasal cannula 210 may be worn by placing supply tubes 212 and 214 over each ear of the user, and may be secured under the chin to fit comfortably relative to the nose of a user. The process for positioning a nasal cannula on a user will be understood to one of ordinary skill in the art from the description herein.

In step 320, a flow of heated and humidified gas is provided. In an exemplary embodiment, source 220 of heated and humidified gas is provided to the supply tube 212 and along the first flow path of nasal cannulas 210. As stated above, nasal cannula 210 may include a connector 216 coupled to the inlet of supply tube 212. Thus, step 320 may comprise coupling source 220 to connector 216 of nasal cannula 210.

In step 330, a flow of aerosolized medicament is provided. In an exemplary embodiment, source 230 of aerosolized medicament is provided to the supply tube 214 and along the second flow path of nasal cannula 210. The flow of aerosolized medicament from source 230 is provided separately from the flow of heated and humidified gas from source 220. As stated above, nasal cannula 210 may include a connector 218 coupled to the inlet of supply tube 214. Thus, step 330 may comprise coupling source 230 to connector 218 of nasal cannula 210. It may be particularly desirable that steps 320 and 330 be performed simultaneously.

It will be understood to one of ordinary skill in the art that method 300 is not limited to the above steps. Method 300 may include additional or alternative steps. Examples of such steps are provided below.

Nasal cannula 210 may optionally include a septum, as described above with respect to nasal cannula 100. Where nasal cannula 210 does not include a septum, method 300 may include the step of providing both the flow of heated and humidified gas and the flow of aerosolized medicament to each of the nasal prongs of nasal cannula 210. Where nasal cannula 210 includes a septum, method 300 may include the step of providing the flow of heated and humidified gas to a first nasal prong of nasal cannula 210 and providing the flow of aerosolized medicament to the second nasal prong of nasal cannula 210.

The exemplary apparatus, systems, and methods described herein may provide advantages over conventional respiratory therapy as set forth below. Conventional respiratory therapy systems were not previously able to deliver warm aerosolized medication combined with a continuous high flow supplemental heated humidified breathing gas via a nasal cannula. Such combined therapy of warm aerosolized medicaments and high flow therapy for patients experiencing stressful respiratory episodes in acute respiratory compromise may prove a comfortable, safe and effective technique in decreasing bronchial responsiveness while maintaining delivery of high FiO2 to improve oxygen saturation level and decrease the work of breathing.

The exemplary cannula described herein is operable to deliver airflow simultaneously from two different gas sources for inhalation therapy. The dual inlet cannula prevents the high gas pressure of the heated and humidified gas source from overcoming, and as a result significantly slowing or completely cutting off, the medicament from the aerosolized medicament source.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A nasal cannula for administering respiratory therapy to a user, the cannula comprising:
a first supply tube having a first inlet to receive a first flow from a first flow source and a first outlet;
a second supply tube having a second inlet to receive a second flow from a second flow source and a second outlet distinct from the first inlet and the first outlet, respectively;
a cannula body having first and second ends, the first end coupled to the first outlet and the second end coupled to the second outlet;
first and second nasal prongs extending from the cannula body;
a first flow path extending from the first supply tube through the cannula body to the first nasal prong to deliver at least a portion of the first flow through the first nasal prong; and
a second flow path extending from the second supply tube through the cannula body to the second nasal prong to deliver at least a portion of the second flow through the second nasal prong.

2. The nasal cannula of claim 1, further comprising:
a first connector coupled to the first inlet; and
a second connector distinct from the first connector coupled to the second inlet.

3. The nasal cannula of claim 1, wherein the first flow path further extends to the second nasal prong, and the second flow path further extends to the first nasal prong.

4. The nasal cannula of claim 1, wherein the cannula body further comprises a septum between the first and second nasal prongs, the septum blocking the first flow path from extending to the second nasal prong and blocking the second flow path from extending to the first nasal prong.

5. The nasal cannula of claim 1, wherein:
the cannula body comprises a tubular portion;
the first and second nasal prongs extend in a substantially radial direction from the tubular portion;
the first supply tube is coupled to a first axial end of the nasal cannula; and
the second supply tube is coupled to a second axial end of the nasal cannula opposite the first axial end.

6. A system for administering respiratory therapy to a user using the nasal cannula of claim 1, the system comprising:
the nasal cannula;
the first flow source; and
the second flow source.

7. The system of claim 6, wherein the first flow source comprises a source of heated and humidified gas coupled to the first inlet to provide the heated and humidified gas along the first flow path; and
the second flow source comprises a source of aerosolized medicament coupled to the second inlet to provide the aerosolized medicament along the second flow path.

8. The system of claim 6, further comprising:
a first connector coupled to the first inlet; and
a second connector distinct from the first connector coupled to the second inlet.

9. The system of claim 8, further comprising:
a delivery tube coupled to the first connector to provide the heated and humidified gas from the first flow source to the first inlet; and
a corrugated tube coupled to the second connector to provide the aerosolized medicament from the second flow source to the second inlet.

10. The system of claim 9, further comprising:
a circuit connector for coupling the corrugated tube to the second connector.

11. The nasal cannula of claim 6, wherein the first flow path further extends to the second nasal prong, and the second flow path further extends to the first nasal prong.

12. The nasal cannula of claim 6, wherein the cannula body further comprises a septum between the first and second nasal prongs, the septum blocking the first flow path from extending to the second nasal prong and blocking the second flow path from extending to the first nasal prong.

13. A method for administering respiratory therapy to a user using the nasal cannula of claim 1, the method comprising the steps of:
positioning the nasal cannula on the user such that the first and second nasal prongs are positioned in respective nostrils of the user;
providing a flow of heated and humidified gas along the first flow path; and
providing a flow of aerosolized medicament separate from the flow of heated and humidified gas along the second flow path.

14. The method of claim 13,
wherein the nasal cannula further comprises a first connector coupled to the first inlet and a second connector distinct from the first connector coupled to the second inlet;
wherein the step of providing the flow of heated and humidified gas comprises coupling a source of heated and humidified gas to the first connector; and
wherein the step of providing a flow of aerosolized medicament comprises coupling a source of aerosolized medicament to the second connector.

15. The method of claim 13,
wherein the step of providing a flow of heated and humidified gas comprises providing a flow of heated and humidified gas to the first and second nasal prongs of the nasal cannula; and
wherein the step of providing a flow of aerosolized medicament comprises providing a flow of aerosolized medicament to the first and second nasal prongs of the nasal cannula.

16. The method of claim 13,
wherein the cannula body further comprises a septum between the first and second nasal prongs;
wherein the step of providing a flow of heated and humidified gas comprises providing a flow of heated and humidified gas to the first nasal prong of the nasal cannula; and
wherein the step of providing a flow of aerosolized medicament comprises providing a flow of aerosolized medicament to the second nasal prong of the nasal cannula.

17. The method of claim 13, wherein the step of providing the flow of heated and humidified gas occurs simultaneously with the step of providing the flow of aerosolized medicament.
